(19) United States
(12) Patent Application Publication
(10) Pub. No.: US 2021/0338963 A1
(43) Pub. Date: Nov. 4, 2021

(54) MEDICAL DEVICE RESPIRATOR

(71) Applicant: Ator Labs, Inc., Panama City Beach, FL (US)

(72) Inventors: Robert Nickell Moran, Panama City Beach, FL (US); Christopher Whittle, Panama City Beach, FL (US); Francisco Hernandez, Panama City Beach, FL (US)

(73) Assignee: Ator Labs, Inc., Panama City Beach, FL (US)

(21) Appl. No.: 17/306,720

(22) Filed: May 3, 2021

Related U.S. Application Data
(60) Provisional application No. 63/019,246, filed on May 1, 2020.

(51) Int. Cl. A61M 16/08; A61M 16/20; A61M 16/10; A61M 16/06

(52) U.S. Cl. CPC A61M 16/0816; A61M 16/208; A61M 16/06; A61M 16/1065; A61M 16/1005

(57) ABSTRACT

A medical device respirator comprising a valve assembly attached to a mask at a mask juncture; an inhale tube attached to the valve assembly and attached to an inhale filter; an exhale tube attached to the valve assembly; an inhale check valve disposed at an attachment juncture between the inhale tube and the valve assembly; an exhale check valve disposed at an attachment juncture between the valve assembly and the exhale tube; wherein inhaled air passes through the inhale filter, the inhale tube, the inhale check valve, the valve assembly, the mask juncture, the mask and into the user; and exhaled air passes through the mask, the mask juncture, the valve assembly, the exhale check valve, and the exhale tube.

### CROSS-REFERENCE TO RELATED APPLICATIONS

This application is a non-provisional application tracing priority and claiming benefit to U.S. Provisional Application No. 63/019,246, filed May 1, 2020, entitled “MEDICAL DEVICE RESPIRATOR”, the entirety of which is expressly incorporated herein by reference.

### FIELD

This invention relates generally to the field of respirators, and more particularly embodiments of the invention relate to adapters for using existing masks and filters for creating respirators.

### BRIEF SUMMARY

Embodiments of the present invention address the above needs and/or achieve other advantages by providing apparatuses and methods for emergency ventilation.

Embodiments of the invention provide a medical device respirator comprising a valve assembly for removably attaching to a mask at a mask juncture; an inhale tube removably attached to the valve assembly and removably attached to an inhale filter; an exhale tube removably attached to the valve assembly; an inhale check valve disposed at an attachment juncture between the inhale tube and the valve assembly; an exhale check valve disposed at an attachment juncture between the valve assembly and the exhale tube; wherein during user inhale, inhaled air passes through the inhale filter, through the inhale tube, through the inhale check valve, through the valve assembly, through the mask juncture, through the mask and into the user; and wherein during user exhale, exhaled air passes through the mask, through the mask juncture, through the valve assembly, through the exhale check valve, and through the exhale tube.

In some embodiments, the exhale tube is further removably attached to an exhale filter and wherein during user exhale, the exhaled air further passes through the exhale filter.

In some embodiments, the inhale check valve is configured to allow air flow only during user inhale. In some embodiments, the exhale check valve is configured to allow air flow only during user exhale.

According to embodiments of the invention, a medical device respirator includes a valve assembly for removably attaching to a mask at a mask juncture; an inhale check valve disposed at a first attachment juncture of the valve assembly; an exhale check valve disposed at a second attachment juncture of the valve assembly; wherein during user inhale, inhaled air passes through the inhale filter, through the inhale check valve, through the valve assembly, through the mask juncture, through the mask and into the user; and wherein during user exhale, exhaled air passes through the mask, through the mask juncture, through the valve assembly, and through the exhale check valve.

In some embodiments, the exhale tube is further removably attached to an exhale filter and wherein during user exhale, the exhaled air further passes through the exhale filter.

In some embodiments, the inhale check valve is configured to allow air flow only during user inhale. In some embodiments, the exhale check valve is configured to allow air flow only during user exhale.

According to some embodiments of the invention, a method for enabling respiration using a medical device respirator includes removably attaching a valve assembly to a mask at a mask juncture; removably attaching an inhale tube to the valve assembly and an inhale filter; removably attaching an exhale tube to the valve assembly; passing inhaled air, during a user inhale, through an inhale filter, through the inhale tube, through an inhale check valve disposed at an attachment juncture between the inhale tube and the valve assembly, through the valve assembly, through the mask juncture, through the mask and into the user; passing exhaled air, during user exhale, through the mask, through the mask juncture, through the valve assembly, through an exhale check valve disposed at an attachment juncture between the valve assembly and the exhale valve, and through the exhale tube.

In some embodiments, the method also includes during user exhale, passing air through an exhale filter removably attached to the exhale tube.

In some embodiments, the inhale check valve is configured to allow air flow only during user inhale. In some embodiments, the exhale check valve is configured to allow air flow only during user exhale.

In some embodiments, the medical device respirator also includes an oxygen port disposed on the mask or the cover and configured for receiving a predetermined flow of oxygenated air for the user. In some such embodiments, the oxygenated air is mixed with air inhaled through the inhale filter and the mixed air flows into the user.

In some embodiments, the method also includes inputting oxygenated air, through an oxygen port, to the respirator at least during user inhale, the oxygenated air for mixing with the inhaled air for inhalation by the user.

In some embodiments, the oxygen port is disposed in a cover, in the mask, in the valve assembly, or in a filter housing, external to an inhale check valve.

In some embodiments, the method also includes removably attaching an inhale tube to the valve assembly and an inhale filter; and removably attaching an exhale tube to the valve assembly, wherein the exhale tube is the output.

The features, functions, and advantages that have been discussed may be achieved independently in various embodiments of the present invention or may be combined in yet other embodiments, further details of which can be seen with reference to the following description and drawings.

### BRIEF DESCRIPTION OF THE SEVERAL VIEWS OF THE DRAWINGS

Having thus described embodiments of the invention in general terms, reference will now be made to the accompanying drawings, wherein:

FIGS. 1A, 1B, and 1C illustrate a medical device respirator according to embodiments of the invention;

FIGS. 2A and 2B illustrate a medical device respirator according to other embodiments of the invention;

FIGS. 3A and 3B illustrate a medical device respirator according to other embodiments of the invention;

FIGS. 4A and 4B illustrate a medical device respirator according to other embodiments of the invention;

FIGS. 5A, 5B, and 5C illustrates multiple embodiments of a medical device respirator; and

FIGS. 6A, 6B, and 6C illustrates a medical device respirator according to other embodiments of the invention.

### DETAILED DESCRIPTION OF EMBODIMENTS OF THE INVENTION

Embodiments of the present invention will now be described more fully hereinafter with reference to the accompanying drawings, in which some, but not all, embodiments of the invention are shown. Indeed, the invention may be embodied in many different forms and should not be construed as limited to the embodiments set forth herein; rather, these embodiments are provided so that this disclosure will satisfy applicable legal requirements. Like numbers refer to like elements throughout.

The present invention provides an medical device respirator comprising a valve assembly for removably attaching to a mask at a mask juncture; an inhale tube removably attached to the valve assembly and removably attached to an inhale filter; an exhale tube removably attached to the valve assembly; an inhale check valve disposed at an attachment juncture between the inhale tube and the valve assembly; an exhale check valve disposed at an attachment juncture between the valve assembly and the exhale tube; wherein during user inhale, inhaled air passes through the inhale filter, through the inhale tube, through the inhale check valve, through the valve assembly, through the mask juncture, through the mask and into the user; and wherein during user exhale, exhaled air passes through the mask, through the mask juncture, through the valve assembly, through the exhale check valve, and through the exhale tube. In some embodiments, the exhale tube is further removably attached to an exhale filter and wherein during user exhale, the exhaled air further passes through the exhale filter. In some embodiments, the medical device respirator includes the inhale filter and the exhale filter.

Embodiments of the invention provide for a non-rebreathing valve adapters, half masks, and filters that can provide bacterial and viral respiratory protection to clinicians and civilians in all environments. In some embodiments, the adapter connects to Smith's Medical anesthesia mask, or Hans Rudolph's 6450 series Disposable V-2 mask, and to A-M Systems VB MAX 44 mm filters. The combination of components allows common materials to become significant respiratory protection for civilian and medical personnel.

In some embodiments, a re-purposed two-way non-rebreathing valve assembly may be used as the connection assembly between anesthesia masks and patient breathing circuit filters. The valve assembly allows the filters to be placed on the inhalation and exhalation sides, limiting exposure of the wearer and the general public to virus such as COVID-19 or bacteria.

As a means of respiratory support, the product may be effective to prevent inhalation of COVID-19 virus. Although the product can be operated by the general public, it is intended for use by licensed or qualified medical providers. It is lightweight, highly portable and can be deployed in a variety of environments, including, but not limited to, intensive care units, inpatient units, emergency rooms, alternate care sites, field hospitals, ambulances, grocery stores, and gas stations—literally anywhere where people require respiratory protection from the COVID-19 virus.

Currently, an outbreak of respiratory disease “Coronavirus Disease 2019” (“COVID-19”) caused by “severe acute respiratory syndrome coronavirus 2” (“SARS-CoV-2”) has brought into stark reality the need for additional patient ventilators. On Jan. 31, 2020, the United States Department of Health and Human Services issued a declaration of public health emergency related to COVID-19. On Mar. 13, 2020, President Trump declared a national emergency in response to COVID-19.

SARS-CoV-2 has demonstrated the capability to spread rapidly, leading to significant impacts on health care systems and causing societal disruption. The potential public health threat posed by COVID-19 is high in the United States and across the world. COVID-19 is associated with severe disease that requires intensive care in approximately 5% of confirmed infections. Given how common the disease is becoming, as in prior major severe acute respiratory infection outbreaks, respiratory protection is an integral component of the global response.

It has been well-documented that there is a global shortage of N95 respirators, FFRs, and surgical masks. Many health care providers have been unable to source these products on the open market. Health care providers are re-using contaminated N95s.

Embodiments of the invention provide for a low cost, easily decontaminated and/or disposable, filtered face surgical respirator with 99.99% viral and bacterial filtering. It is used by those requiring respiratory protection against exposure to COVID-19 or other viral or bacterial agents.

Embodiments of the invention may use filters such as A-M Systems VB MAX filter p/n 810000; disposable; single-use; designed to prevent bacterial/viral contamination; suitable for adults and children; FDA 510(k) K000654; user defined requirements; can be used only on inhalation outlet or on both inhalation and exhalation outlets.

Embodiments of the invention may use for a mask: Hans Rudolph 6450 Series Disposable mask with disposable head band or Smiths Medical anesthesia mask with elastic head straps p/n: 5425r/5255/5248 (Provided by either hospital or distributor).

Embodiments of the invention may use a two way non-rebreathing assembly. A two-way non-rebreathing assembly is comprised of simple components: (1) inhalation check valve; (2) exhalation check valve and (3) mask connection/valve body. The assembly has less than 30 ml H₂O of dead space. These three components are supported by the valve body. This valve is connected at the inhalation and exhalation ports. The ends of the valve allow for the connection of various ventilator/spirometry filters.

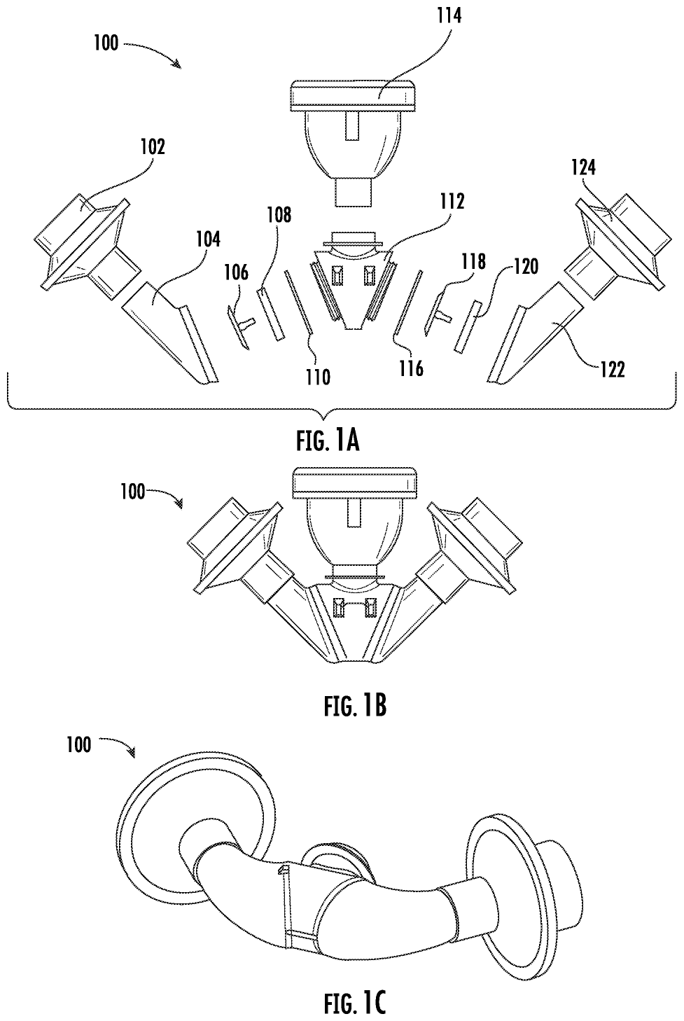

Referring to FIGS. 1A, 1B, and 1C, an embodiment of a medical device respirator 100 is shown. An input filter 102 receives air as a patient inhales. The air passes through an inhale tube 104 and through a one-way inhale check valve 106. The inhale check valve is disposed inside the inhale tube and secured with a spacer 108 and ring 110 when the inhale tube is attached to the valve assembly 112. The valve assembly is attached to a mask 114 at a mask juncture. As the user or patient inhales, air is allowed to pass through inhale check valve 106 through the mask and into the patient. During exhalation, air flows out from the patient, through the mask 114, through the valve assembly 112, through the exhale check valve 118, through the exhale tube 122 and through the exhale filter 124. In some embodiments, the respirator does not include an exhale filter 124 and/or an inhale filter 102. The exhale check valve 118 is secured inside the exhale tube 122 by a spacer 120 and a ring 116 when the exhale tube is attached to the valve assembly 112. For clarity, during inhalation, air is prevented from flowing from the outside through the exhale check valve 118, and similarly, during exhalation, air is prevented from flowing from inside the valve assembly 112 through the inhale check valve 106.

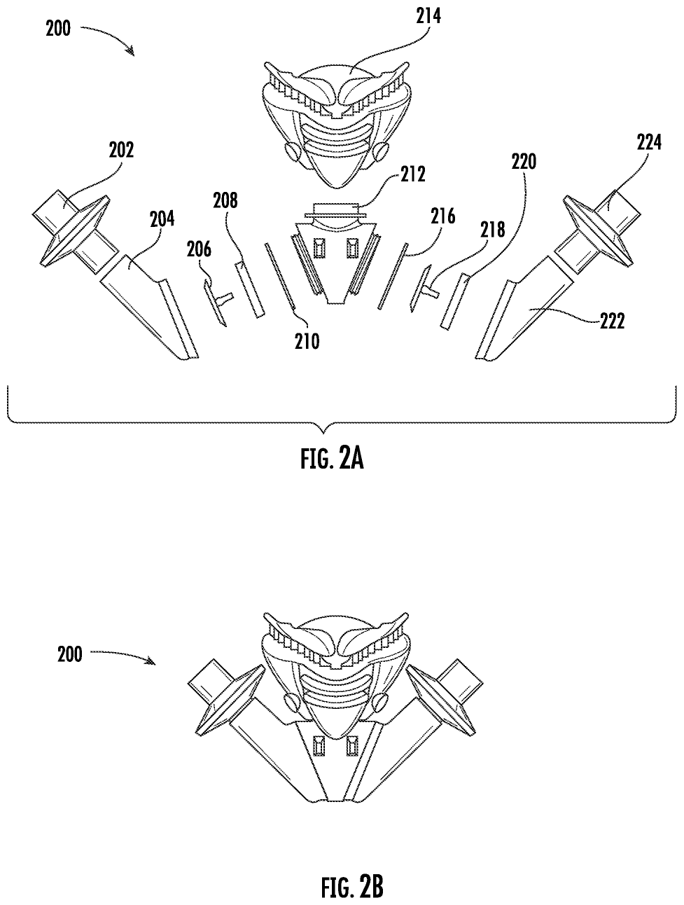

Referring now to FIGS. 2A and 2B, another embodiment of the medical device respirator 200 is shown. In this embodiment, during inhalation, air flows from outside through an inhale filter 202 attached to an inhale tube 204 and through an inhale check valve 206, which is secured in the inhale tube 204 with a spacer 208 and a ring 210 when the inhale tube 204 is attached to the valve assembly 212. The air flows through the valve assembly and into the patient through the mask 214. During exhalation, air flows out from the patient, through the mask 214, through the valve assembly 212, through the exhale check valve 218, which is secured inside the exhale tube 222 using a spacer 220 and a ring 216 when the exhale tube 222 is attached to the valve assembly 212. The exhale tube is attached to an exhale filter 224. In different embodiments, the inhale and/or the exhale filters are optional.

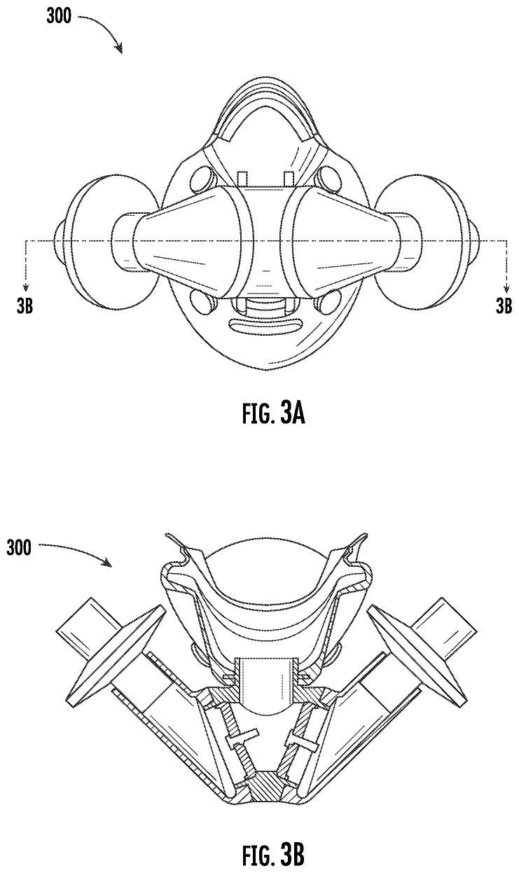

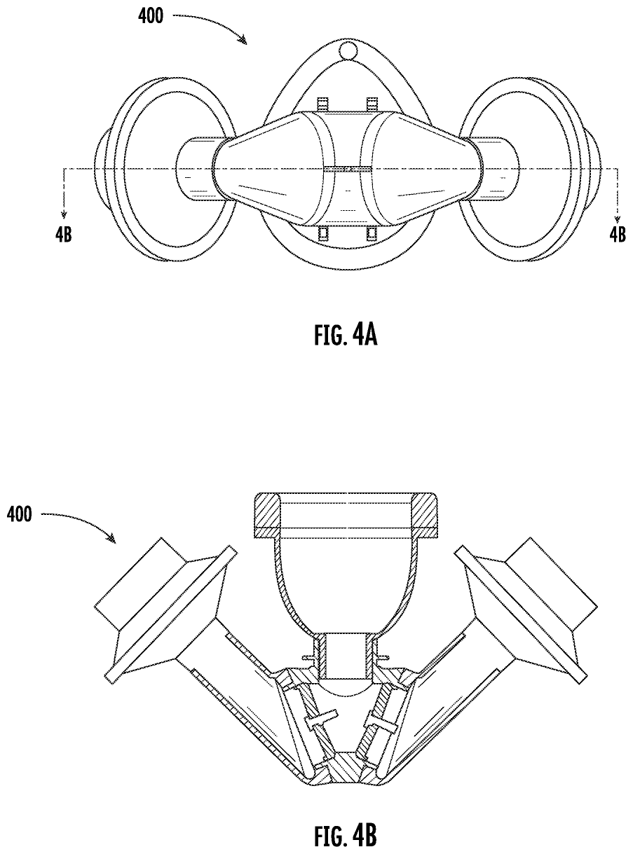

Referring now to FIGS. 3A and 3B another embodiment of a medical device respirator 300 is shown having similar functionality as those discussed above. Referring to FIGS. 4A and 4B, another embodiment of a medical device respirator 400 is shown having similar functionality as those discussed above.

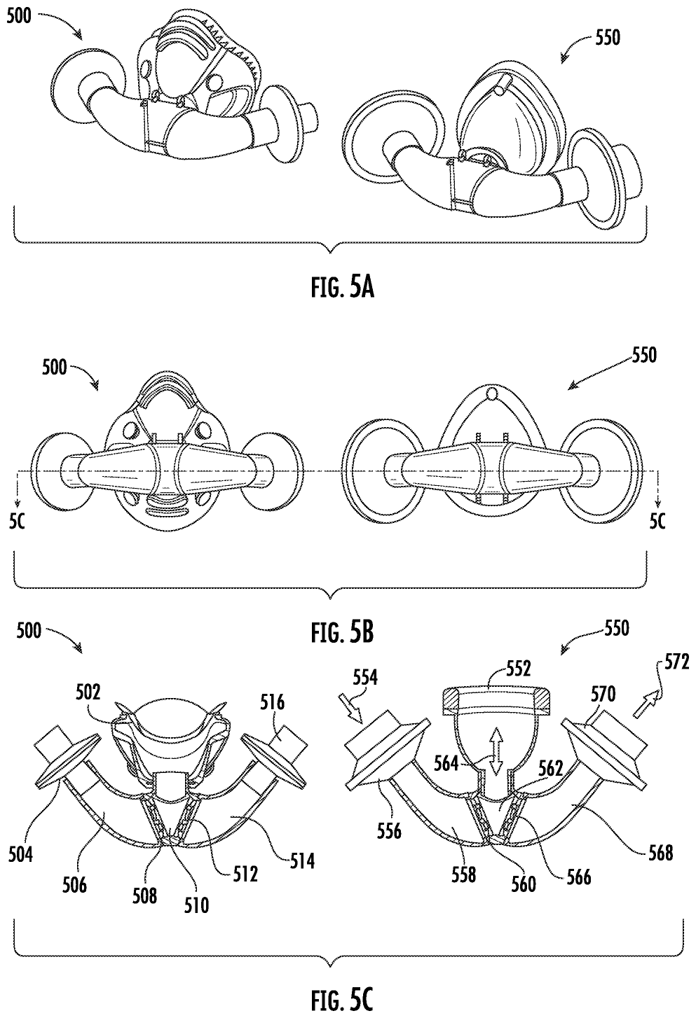

Referring now to FIGS. 5A, 5B, and 5C, two embodiments of the medical device respirator 500 and 550 are shown side-by-side.

In respirator 500, during inhalation, air flows from outside through an inhale filter 504 attached to an inhale tube 506 and through an inhale check valve 508, which is secured in the inhale tube 506 with a spacer and a ring when the inhale tube 506 is attached to the valve assembly 510. The air flows through the valve assembly 510 and into the patient through the mask 502. During exhalation, air flows out from the patient, through the mask 502, through the valve assembly 510, through the exhale check valve 512, which is secured inside the exhale tube 514 using a spacer and a ring when the exhale tube 514 is attached to the valve assembly 510. The exhale tube is attached to an exhale filter 516. In different embodiments, the inhale and/or the exhale filters are optional.

In respirator 550, during inhalation, air flows in the direction of arrow 554 from outside through an inhale filter 556 attached to an inhale tube 558 and through an inhale check valve 560, which is secured in the inhale tube 558 with a spacer and a ring when the inhale tube 506 is attached to the valve assembly 562. The air flows through the valve assembly 562 and into the patient through the mask 552 as illustrated by arrow 564. During exhalation, air flows out from the patient, also as illustrated by arrow 564, through the mask 502, through the valve assembly 510, through the exhale check valve 512, which is secured inside the exhale tube 514 using a spacer and a ring when the exhale tube 514 is attached to the valve assembly 510. The exhaled air flows through the exhale tube 568 attached to an exhale filter 516 in the direction of arrow 572. In different embodiments, the inhale and/or the exhale filters are optional.

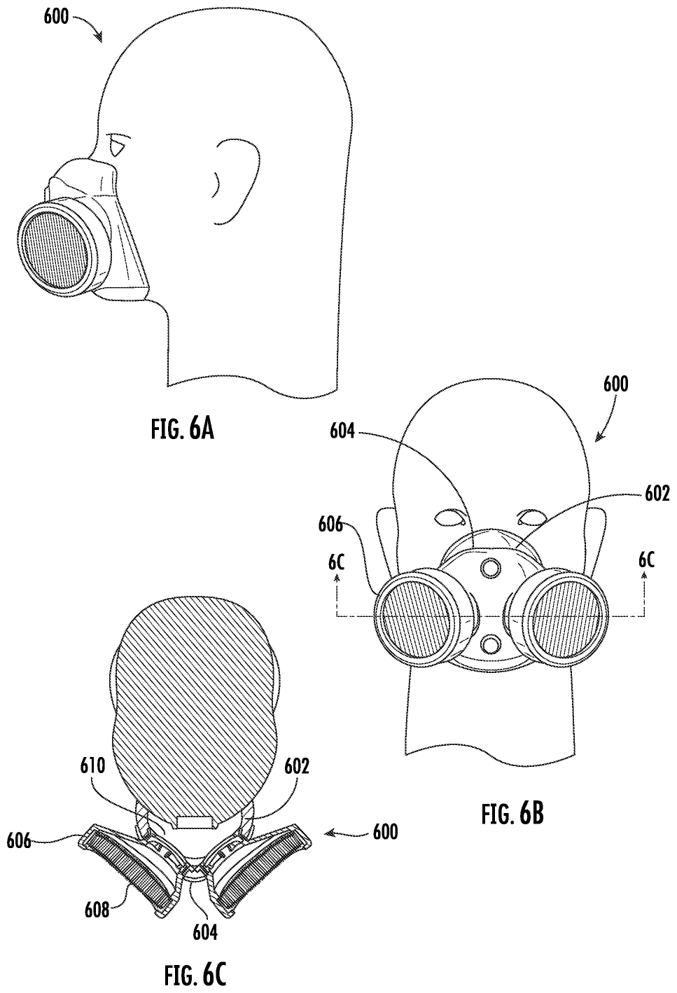

Referring to FIGS. 6A, 6B, and 6C another embodiment of a medical device respirator 600 is illustrated. In this embodiment, an oxygen port is attached to a mask, such as a silicone mask. The oxygen port may enable oxygen to be ported into the cover 604 and/or the mask 602 as the user inhales. For example, at an oxygen input rate of 3 lpm oxygen, the average inhaled oxygen content has been measured as 32%. This respirator 600 may function as an infected patient oxygen mask that lowers the viral content of the air in an enclosed space, such as a hospital room. The respirator 600 enables the patient's inhaled air and exhaled air to be filtered by filters 608 in a filter housing 606. As discussed with other embodiments, the respirator includes an inhale check valve such as check valve 610 and exhale check valve. In some embodiments, the oxygen port is disposed on the cover 604 and in other embodiments it is disposed on the mask 602, and in some embodiments, the oxygen port passes through both the cover and the mask to provide oxygenated air to the inside of the mask. The oxygenated air, in some embodiments, is mixed with air inhaled through filter 608 and then provided through the mask to the user. Exhaled air is output through the opposite filter. The flow of oxygenated air may be controlled, such as through feedback control, or may be constant such that extra oxygenated air, or oxygenated air input during an exhale phase is simply output through the exhale check valve and exhale filter along with the user's exhaled air. In another embodiment, the oxygen port is disposed on the inhale filter cover external to the inhale check valve such that oxygen is pulled into the mask along with external air for inhalation.

The embodiment of FIGS. 6A, 6B, and 6C is applicable to patients with congestive heart failure, COPD, Cystic Fibrosis, and any-other “lung impairment” that requires the ambulatory patient to receive supplemental oxygen.

While certain exemplary embodiments have been described and shown in the accompanying drawings, it is to be understood that such embodiments are merely illustrative of, and not restrictive on, the broad invention, and that this invention not be limited to the specific constructions and arrangements shown and described, since various other changes, combinations, omissions, modifications and substitutions, in addition to those set forth in the above paragraphs, are possible. Those skilled in the art will appreciate that various adaptations, modifications, and combinations of the just described embodiments can be configured without departing from the scope and spirit of the invention. Therefore, it is to be understood that, within the scope of the appended claims, the invention may be practiced other than as specifically described herein.

What is claimed is:

1. A medical device respirator comprising:
a valve assembly for removably attaching to a mask at a mask juncture;
an inhale tube removably attached to the valve assembly and removably attached to an inhale filter;
an exhale tube removably attached to the valve assembly;
an inhale check valve disposed at an attachment juncture between the inhale tube and the valve assembly;
an exhale check valve disposed at an attachment juncture between the valve assembly and the exhale tube;
wherein during user inhale, inhaled air passes through the inhale filter, through the inhale tube, through the inhale check valve, through the valve assembly, through the mask juncture, through the mask and into the user;
wherein during user exhale, exhaled air passes through the mask, through the mask juncture, through the valve assembly, through the exhale check valve, and through the exhale tube.

2. The medical device respirator of claim 1, wherein the exhale tube is further removably attached to an exhale filter and wherein during user exhale, the exhaled air further passes through the exhale filter.

3. The medical device respirator of claim 1, wherein the inhale check valve is configured to allow air flow only during user inhale.

4. The medical device respirator of claim 1, wherein the exhale check valve is configured to allow air flow only during user exhale.

5. A medical device respirator comprising:
a valve assembly for removably attaching to a mask at a mask juncture;
an inhale check valve disposed at a first attachment juncture of the valve assembly;
an exhale check valve disposed at a second attachment juncture of the valve assembly;
wherein during user inhale, inhaled air passes through the inhale filter, through the inhale check valve, through the valve assembly, through the mask juncture, through the mask and into the user; and
wherein during user exhale, exhaled air passes through the mask, through the mask juncture, through the valve assembly, and through the exhale check valve.

6. The medical device respirator of claim 5, wherein the exhale tube is further removably attached to an exhale filter and wherein during user exhale, the exhaled air further passes through the exhale filter.

7. The medical device respirator of claim 5, wherein the inhale check valve is configured to allow air flow only during user inhale.

8. The medical device respirator of claim 5, wherein the exhale check valve is configured to allow air flow only during user exhale.

9. The medical device respirator of claim 5, further comprising an oxygen port disposed on the mask or the cover and configured for receiving a predetermined flow of oxygenated air for the user.

10. The medical device respirator of claim 9, wherein the oxygenated air is mixed with air inhaled through the inhale filter and the mixed air flows into the user.

11. A method for enabling respiration using a medical device respirator, the method comprising:
removably attaching a valve assembly to a mask at a mask juncture;
passing inhaled air, during a user inhale, through an inhale filter, through an inhale check valve through the valve assembly, through the mask juncture, through the mask and into the user;
passing exhaled air, during user exhale, through the mask, through the mask juncture, through the valve assembly, through an exhale check valve, and through an output.

12. The method of claim 9, further comprising, during user exhale, passing air through an exhale filter removably attached to the output.

13. The method of claim 9, wherein the inhale check valve is configured to allow air flow only during user inhale.

14. The method of claim 9, wherein the exhale check valve is configured to allow air flow only during user exhale.

15. The method of claim 9, further comprising:
inputting oxygenated air, through an oxygen port, to the respirator at least during user inhale, the oxygenated air for mixing with the inhaled air for inhalation by the user.

16. The method of claim 15, wherein the oxygen port is disposed in a cover.

17. The method of claim 15, wherein the oxygen port is disposed in the mask.

18. The method of claim 15, wherein the oxygen port is disposed in the valve assembly.

19. The method of claim 15, wherein the oxygen port is disposed in a filter housing, external to an inhale check valve.

20. The method of claim 15, further comprising:
removably attaching an inhale tube to the valve assembly and an inhale filter; and
removably attaching an exhale tube to the valve assembly, wherein the exhale tube is the output.
